(19) United States
(12) Patent Application Publication
(10) Pub. No.: US 2016/0082230 A1
(43) Pub. Date: Mar. 24, 2016

(54) COMPLIANT SLEEVE FOR VASCULAR BALLOON

(71) Applicant: Cook Medical Technologies LLC, Bloomington, IN (US)

(72) Inventors: Thomas W. McGhie, Bloomington, IN (US); Alyson M. McGhie, Bloomington, IN (US); Christopher B. Crabtree, Bloomington, IN (US)

(73) Assignee: Cook Medical Technologies LLC, Bloomington, IN (US)

(21) Appl. No.: 14/857,178

(22) Filed: Sep. 17, 2015

Related U.S. Application Data
(60) Provisional application No. 62/053,373, filed on Sep. 22, 2014.

(51) Int. Cl. A61M 25/10; A61F 2/844; A61F 2/966; A61F 2/958

(52) U.S. Cl. CPC A61M 25/10184; A61M 25/104; A61F 2/958; A61F 2/844; A61F 2/966; A61M 2025/1081; A61B 17/12136

(57) ABSTRACT

The present disclosure relates to vascular balloon catheters having one or more sleeves disposed around at least a portion of the balloon. The balloon may be non-compliant or semi-compliant and its behavior may be modified by using the sleeve. The behavior of the balloon may be modified in any manner by the sleeve where a variable length of the balloon expands before the entire length of the balloon expands.

### BACKGROUND

1. Field of the Invention

The present disclosure generally relates to medical devices. More particularly, the disclosure relates to medical balloons comprising compliant sleeves.

2. Description of the Related Art

Balloon angioplasty is a widely used procedure for expanding constricted body passageways, such as arteries and other blood vessels, or various ducts (e.g., of the biliary system). In an angioplasty procedure, an uninflated angioplasty balloon attached to a catheter is delivered to a constricted region of a body passageway. Once the balloon is in position at the constricted region, fluid is injected through a lumen of the catheter and into the balloon. The balloon consequently inflates and exerts pressure against the constricted region to expand the passageway. After use, the balloon is collapsed, and the catheter is withdrawn.

Although treatment of constricted arteries in the vasculature is one common example where balloon catheters are used, this is only one example of how balloon catheters may be used and many other uses are possible. For example, balloon catheters may be used to deploy a stent to a target location in the body.

Regardless of the intended use of the balloon catheter, there are circumstances where it would be advantageous for only a portion of the balloon to expand or for the balloon to expand in a step-wise manner, for example. Conventional balloons, however, are generally incapable of this portion-wise or step-wise inflation.

### BRIEF SUMMARY

In one embodiment, a system for modifying the behavior of a vascular balloon is disclosed. The system comprises a shaft having a distal end, a proximal end, and an expandable balloon mounted at the distal end of the shaft. The shaft has a lumen extending therethrough, in fluid communication with an interior region of the balloon, and the balloon comprises a body portion, a distal end portion, and a proximal end portion. A tubular sleeve is disposed around the balloon and a portion of the balloon remains uncovered by the sleeve. The sleeve is adhered to the balloon. In some embodiments, a thickness of a wall of the sleeve is varied along a length of the sleeve.

In another embodiment, a system for modifying the behavior of a vascular balloon is disclosed. The system comprises a shaft having a distal end, a proximal end, and an expandable balloon mounted at the distal end of the shaft. The shaft has a lumen extending therethrough, in fluid communication with an interior region of the balloon, and the balloon comprises a body portion, a distal end portion, and a proximal end portion. A tubular sleeve is disposed around the body portion of the balloon and the distal portion of the balloon and the proximal portion of the balloon remain uncovered by the sleeve.

In an additional embodiment, a system for modifying the behavior of a vascular balloon is disclosed. The system comprises a shaft having a distal end, a proximal end, and an expandable balloon mounted at the distal end of the shaft. The shaft has a lumen extending therethrough, in fluid communication with an interior region of the balloon, and the balloon comprises a body portion, a distal end portion, and a proximal end portion. A tubular sleeve is disposed around the proximal portion of the balloon and the distal portion of the balloon remains uncovered by the sleeve or a tubular sleeve is disposed around the distal portion of the balloon and the proximal portion of the balloon remains uncovered by the sleeve.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIGS. 1A-1C depict a cross-sectional view of a medical device system showing a balloon being inflated in two steps;

FIGS. 2A-2D depict a cross-sectional view of a medical device system showing a balloon being inflated in three steps;

FIGS. 3A-3B depict a cross-sectional view of a medical device system showing a balloon being inflated in a “dog bone” configuration and a stent expanding in an hourglass configuration;

FIG. 4 depicts a graph showing a pressure v. strain curve;

FIGS. 5A-5D depict a cross-sectional view of a medical device system showing a scheme where a portion of a balloon is inflated, then deflated, then two portions of the balloon are inflated;

FIG. 6 depicts a cross-sectional view of a system including a balloon having two cones and a sleeve disposed on a portion of the balloon;

FIG. 7 depicts a cross-sectional view of a system including a balloon having three cones and a sleeve disposed on a portion of the balloon; and

FIG. 8 depicts a balloon having a proximal end portion, a distal end portion, and a body portion.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly.

The present disclosure relates to vascular or medical balloon catheters comprising one or more sleeves disposed around at least a portion of the balloon. The balloon may be non-compliant or semi-compliant and its behavior may be modified by using the presently disclosed sleeve. Balloon compliance is meant to describe the degree to which a diameter of a balloon changes as a function of pressure. For example, the diameter of a compliant balloon may have greater than about 10% growth, the diameter of a semi-compliant balloon may have between about 5% growth and about 10% growth, and the diameter of a non-compliant balloon may have less than about 5% growth at the maximum rated pressure compared to a minimal or nominal pressure.

As illustrative examples, the behavior of the balloon may be modified by the sleeve in a manner such that the ends of the balloon expand (open) before a middle portion of the balloon, the middle portion of the balloon expands before the end portions, the proximal portion of the balloon expands before the distal portion, the distal portion of the balloon expands before the proximal portion, or any other configuration where one or more lengths of the balloon expand before the entire length of the balloon expands.

As used herein, the phrase “distal end portion of the balloon,” “distal portion of the balloon,” and the like, refers to the portion of the balloon extending distally from the body portion (mid-portion) of the balloon but excludes the leg/neck portion of the distal end of the balloon, which may be attached to the catheter. A distal portion is a portion located furthest from the handle of a catheter. The phrase “proximal end portion of the balloon,” “proximal portion of the balloon,” and the like, refers to the portion of the balloon extending proximally from the body portion (mid-portion) of the balloon but excludes the leg/neck portion of the proximal end of the balloon, which may be attached to a catheter. A proximal portion is a portion located closest to the handle of the catheter.

Thus, in some embodiments, if it is specified that a proximal portion of the balloon comprises a sleeve or is covered by a sleeve, this may refer to the portion of the balloon located proximally of the body portion, or partially overlapping a proximal section of the body portion of the balloon, including a proximal cone/transition portion but excluding a proximal neck/leg portion. However, the sleeve is not required to always cover, or fully cover, the proximal cone. In some embodiments, the sleeve may cover a proximal portion of the body portion, excluding the proximal cone, and in other embodiments, the sleeve may only cover the proximal cone.

Additionally, in some embodiments, if it is specified that a distal portion of the balloon comprises a sleeve or is covered by a sleeve, this refers to the portion of the balloon located distally of the body portion of the balloon, or partially overlapping a distal section of the body portion of the balloon, including the distal cone/transition portion but excluding the distal neck/leg portion. However, as noted above, the sleeve is not required to always cover, or fully cover, the distal cone. In some embodiments, the sleeve may cover a distal portion of the body portion, excluding the distal cone, and in other embodiments, the sleeve may only cover the distal cone.

The present disclosure also relates to systems for modifying the behavior of a vascular/medical balloon. Some of the systems may include a catheter having a catheter shaft with a distal end and a proximal end. The catheter may be any catheter commonly used to carry out medical procedures, such as angioplasty balloon catheters and balloon-expandable stent catheters.

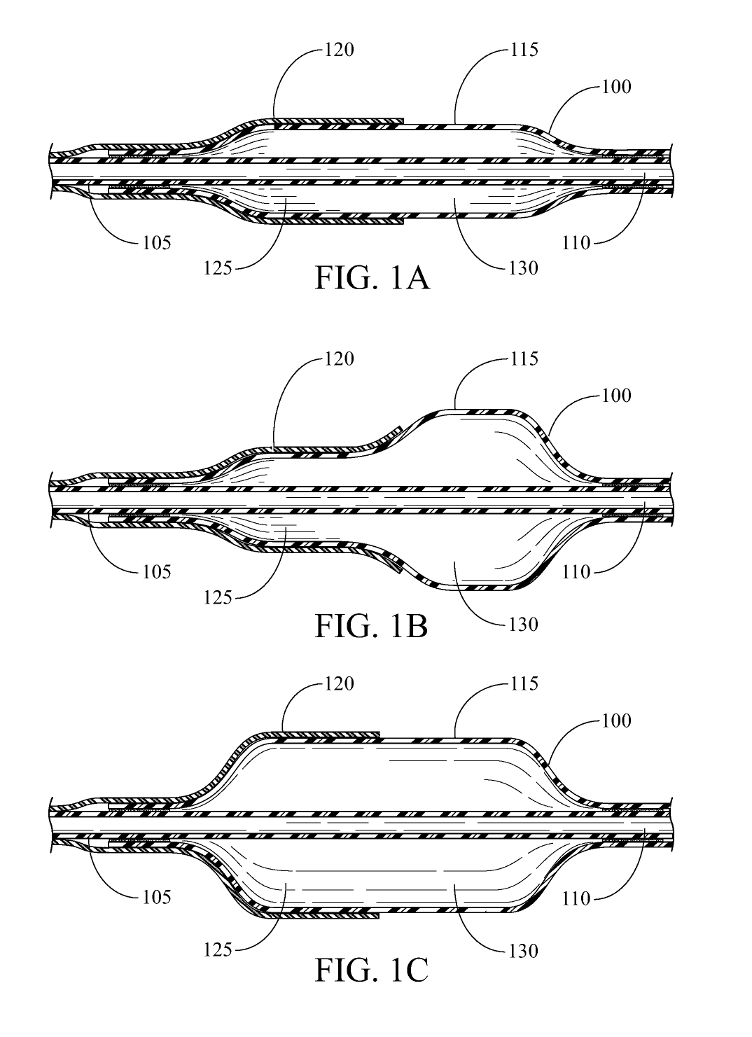

With respect to FIG. 1A, an expandable balloon (100) may be mounted at the distal end of the catheter shaft (105). The catheter shaft comprises a lumen (110) extending therethrough and in fluid communication with an interior region of the balloon. The lumen may be used to supply air or a fluid, such as saline, into the balloon and thereby cause inflation (expansion) of the balloon.

Any known medical balloon may be used in accordance with the present disclosure. Moreover, any known materials, such as elastic materials, may be used to manufacture the balloon. In some embodiments, the balloon comprises a material selected from the group consisting of silicone, polyethyleneterephthalate (PET), polyvinyl chloride, polypropylenes, polyolefins, polyethylenes, polyurethanes, nylons, polyamides, polyamide block copolymers, polyethers, polyesters, latex, natural rubber, synthetic rubbers, elastomers, and any combination thereof.

In some embodiments, such as shown in FIG. 8, the balloon comprises a body portion (860), a distal end portion, and a proximal end portion. The distal end portion of the balloon may comprise a cone (865), which is a portion of the balloon where a transition occurs, such as between a neck/leg (840,845) of the balloon and the mid-portion/body portion (860) of the balloon. The proximal end portion of the balloon may also comprise a cone (870). However, it should be understood that a balloon may include more than two cones, such as three cones, four cones, five cones, etc. With respect to FIG. 7, a balloon is shown comprising a proximal cone (750), a distal cone (755) and an intermediate cone (775).

In some embodiments, the sleeve may be disposed around the balloon such that all portions of the balloon are covered, including the neck portions of the balloon. In other embodiments, such as the embodiment depicted in FIG. 1A, a portion of the balloon (115) may remain uncovered by the sleeve (120). The portion of the balloon that is covered by the sleeve has added resistance to opening during balloon inflation because of the sleeve. For example, with respect to FIG. 1B, if an 8 cm balloon has a sleeve (120) placed over the proximal 4 cm (125), after applying a first pressure to the interior of the balloon, the distal 4 cm (130) can open to the balloon's nominal diameter while the proximal portion remains unopened because the first pressure is not great enough to overcome the resistance of the sleeve, effectively creating a 4 cm balloon. With respect to FIG. 1C, if the user wants the full 8 cm to open/inflate, the pressure may be increased further until the resistance of the sleeve (120) is overcome.

In some embodiments, a stent may be disposed on the sleeve and/or on the balloon. In other embodiments, a plurality of stents may be disposed on the sleeve and/or balloon, such as two stents, three stents, four stents, etc. In one particular embodiment, a stent may be disposed on the sleeve, a portion of the balloon may remain uncovered by the sleeve, and a second stent may be placed around that uncovered portion of the balloon. In the case of a balloon-expanding (BX) stent, the sleeve may be placed between the balloon and the stent.

As noted herein, the sleeve may be disposed around a portion of the balloon such that another portion (or portions) of the balloon remains uncovered. The presently disclosed sleeves may be distinguished from splittable sleeves in that the presently disclosed sleeves circumferentially surround the balloon both in the unexpanded and expanded states of the balloon. Additionally, more than one sleeve, such as two sleeves, may be disposed around the balloon such that portions of the balloon are covered by the sleeve and other portions are not covered by the sleeve. In some embodiments, the sleeve may be bonded or otherwise attached to one end, or both ends, of a catheter shaft. The sleeve may also be adhered to the balloon.

In accordance with the present disclosure, the terms “adhered,” “adhere,” and the like, refer to a configuration where the sleeve is disposed on a portion of the balloon and remains stationary on that portion of the balloon during expansion of the balloon such that the sleeve does not move longitudinally in the proximal or distal direction during expansion. In some embodiments, the sleeve may be adhered to the balloon by using friction forces, bonding, gluing, direct compression, or any combination thereof. In some embodiments, the sleeve may be adhered to the balloon primarily through friction forces but the sleeve may also be bonded to the balloon in one or more locations to further assist in the prevention of longitudinal movement during expansion. In the unexpanded state, the balloon may comprise folds, e.g. the body portion and the cone of the balloon may be folded. That is, in the unexpanded state, the balloon may be provided with a series of longitudinal creases where the balloon wall is folded over itself and wrapped around the shaft in order to achieve a reduced diameter. During expansion of the balloon, the balloon wall unfolds and unwraps in order to achieve a larger diameter with a smooth, unfolded circumference. Folding may be particularly useful for non-compliant and semi-compliant balloons where the wall of the balloon does not significantly stretch when inflation pressure is applied. Thus, in some embodiments, the sleeve may be adhered to the outer surfaces of the folds in the unexpanded state.

The length, thickness, durometer, and other properties of the sleeve may be selected to produce the desired resistance and opening pressure. The wall thickness of the sleeve is not particularly limited and may be selected by the user depending upon the application of the sleeve. In some embodiments, the wall thickness of the sleeve may be constant or uniform. In other embodiments, the wall thickness may vary along the length of the sleeve.

For example, a proximal portion of the sleeve may have a wall thickness that is thinner than the wall thickness of the distal portion of the sleeve. With such a configuration, upon expansion of the balloon comprising the sleeve, the proximal portion of the balloon may open/expand before the distal portion of the balloon. The wall of the sleeve may increase or decrease in thickness (gradually, step-wise, or a combination thereof) along the length of the sleeve. In general, a portion of the sleeve having a thinner wall thickness is less resistant to opening pressure than a portion of the sleeve having a thicker wall thickness. In some embodiments, the sleeve may be radially expandable, longitudinally expandable, or both radially and longitudinally expandable. In some embodiments, the balloon is less resistant to opening pressure than the sleeve.

In accordance with the present disclosure, the thickness of the wall of the sleeve may be, for example, from about 0.005 inches to about 0.015 inches. Thus, in one embodiment, a portion of the sleeve having a thinner wall thickness may comprise a thickness from about 0.005 to less than about 0.01 inches and a portion of the sleeve having a thicker wall thickness may comprise a thickness from greater than about 0.01 to about 0.015 inches. Of course, any desired wall thicknesses may be selected such that one or more portions of the wall of the sleeve are thinner or thicker than one or more other portions of the wall of the sleeve.

The sleeve may comprise an elastic material. In some embodiments, the sleeve comprises more than one material. While any elastic material may be used to manufacture the presently disclosed sleeve, certain illustrative examples of materials that may be used to manufacture the sleeve are as follows: silicone, latex, vulcanized latex, expanded polytetrafluoroethylene (ePTFE), polyolefins, fluoropolymers, noncompliant polyethylene terephthalate (PET), polyimides, nylons, polyamides, polyurethanes, polyesters, polyethylenes, PEBAX, polyesterether copolymers, polyetheresteramide terpolymers.

The sleeve may comprise any one of these materials or the sleeve may comprise any combination of two or more of these materials. In some embodiments, the sleeve may comprise a distal portion, a proximal portion, and a body portion, and each portion of the sleeve may comprise a different material or different combination of materials. Moreover, the distal portion of the sleeve and a distal section of the body portion may comprise one material or combination of materials and the proximal portion and proximal section of the body portion may comprise a different material or different combination of materials. Additionally, two portions of the sleeve may comprise the same material or same combination of materials and a third portion may comprise a different material or different combination of materials.

When different materials are used along the length of the sleeve, this may allow for multiple increments to open in a piece-wise manner because the materials may have different resistances to opening pressure. This is advantageous because it can reduce the number of balloons needing to be stocked at an institution or where the exact length of the balloon needed for a procedure may not be known until mid-way through the procedure.

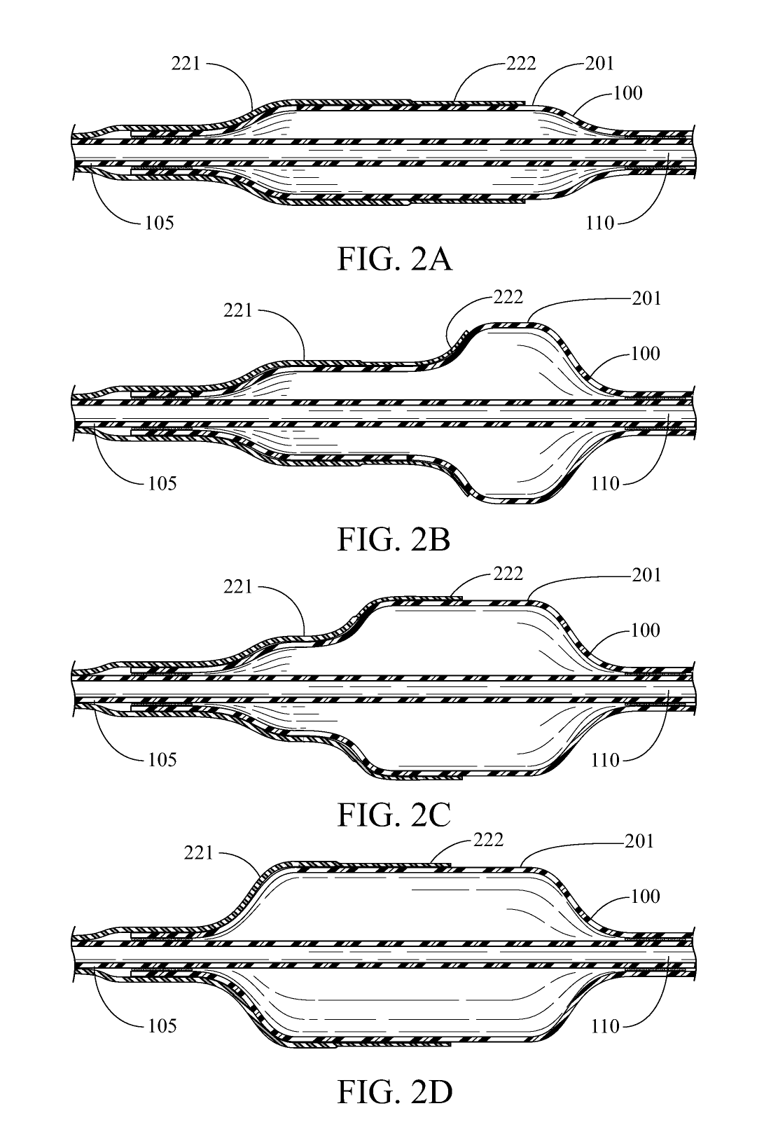

With respect to FIGS. 2A-2D, a sleeve is shown having different wall thickensses (but it is intended that the following description is also applicable if instead the sleeve comprised different materials along its length and had a uniform wall thickness). The sleeve shown in FIGS. 2A-2D comprises two different wall thicknesses; a thin wall thickness (222) and a thicker wall thickness (221). A portion of the balloon (201) remains uncovered by the sleeve. FIG. 2A shows the device before use. During use, pressure is applied from the lumen of the shaft to the balloon and the uncovered portion of the balloon (201) expands, as in FIG. 2B. If the pressure is increased, as in FIG. 2C, the portion of the balloon covered by the thinner wall thickness portion of the sleeve (222) expands. If the pressure is further increased, as in FIG. 2D, the portion of the balloon covered by the thicker wall thickness portion of the sleeve (221) expands. Again, instead of varying the thickness of the wall of the sleeve to allow for the piece-wise opening of the balloon, the sleeve may have a uniform wall thickness but comprise different materials that have different resistances to opening pressures.

For example, if the sleeve had a uniform wall thickness but portion (222) was replaced with a material having a certain resistance to opening pressure and portion (221) was replaced with a material having a higher resistance to opening pressure than the material used for portion (222), the same piece-wise opening of the balloon could be achieved.

When a sleeve is disposed on a balloon in the folded state, the wall thickness (and resistance to opening) of the sleeve is relatively high. Once the pressure of the balloon expanding underneath the sleeve is great enough to cause the sleeve to start expanding, the wall thickness of the sleeve decreases, thereby further reducing the resistance to opening. If the material of the sleeve is selected to have a local minimum in its pressure-strain curve (see FIG. 4), the feedback to pressure allows for a two-state balloon behavior—fully closed below the set pressure or fully open above this set pressure. An example of such a material is latex. This may be desirable versus a long transition pressure of being partially open. In addition to this piece-wise segmented opening behavior, the balloon may behave similar to a modeling balloon, where it is stable at both the uninflated and inflated diameters of the balloon. The inflated length can vary, enabling any length of the underlying balloon to be inflated.

Such an inflation routine could be used, for example, to rapidly deploy multiple overlapping stents or stent grafts on a single balloon. This would be useful in transjugular intrahepatic porto-systemic shunt (TIPS) procedures, where it is common to need an additional few centimiters of stent added to the proximal end of a shunt that proves to be longer than initially estimated. If it turns out that the additional length is not needed, this portion of the balloon and stent disposed thereon may be left uninflated, removed, and disposed of.

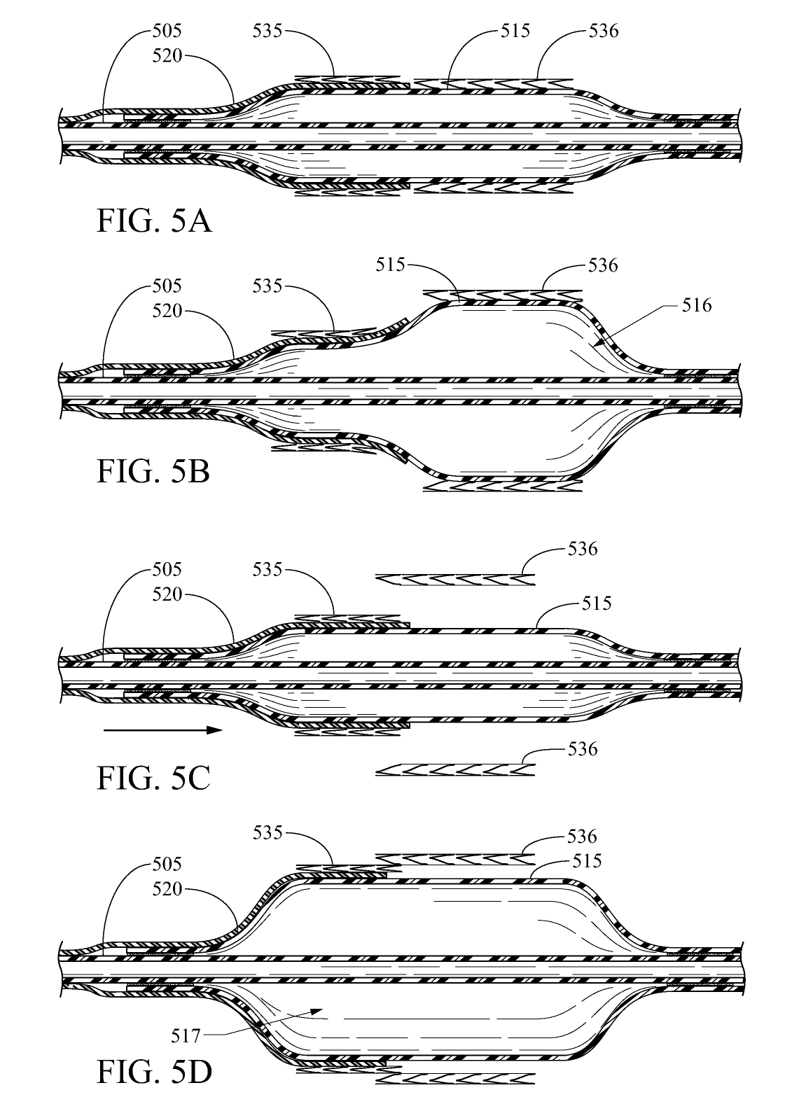

The foregoing routine is generally depicted in FIGS. 5A-5D. FIG. 5A shows the device before use, including a catheter shaft (505), an uninflated balloon (515), a sleeve (520), a first stent (535), and a second stent (536). When a first pressure is applied, the portion of the balloon not covered by the sleeve (516) inflates, thereby expanding the second stent (536), as can be seen in FIG. 5B. The balloon may then be deflated, leaving the second stent (536) in place at the target site, as shown in FIG. 5C. Also shown in FIG. 5C is a repositioning of the catheter, moving it in the distal direction, such that the first stent (535) is partially positioned underneath the second stent (536). After repositioning, a second pressure may be applied to the interior of the balloon, which is greater than the first pressure applied in FIG. 5B, so that the portion of the balloon (517) covered by the sleeve (520) is inflated, thereby expanding the first stent (535), as in FIG. 5D. The balloon may then be deflated once again and removed from the target site, leaving the first and second stents in place. The first stent may be positioned such that, when expanded, it partially overlaps a portion of the second stent or its distal end may be located proximally adjacent to the proximal end of the second stent.

The length of the balloon and the sleeve are not particularly limited and any length may be used. The length of the balloon and sleeve may be selected by the user depending upon the specific application. In some embodiments, the total length of the balloon and/or sleeve may be from about 4 mm to about 120 mm. In other embodiments, the length of the balloon and/or sleeve may be from about 60 mm to about 100 mm.

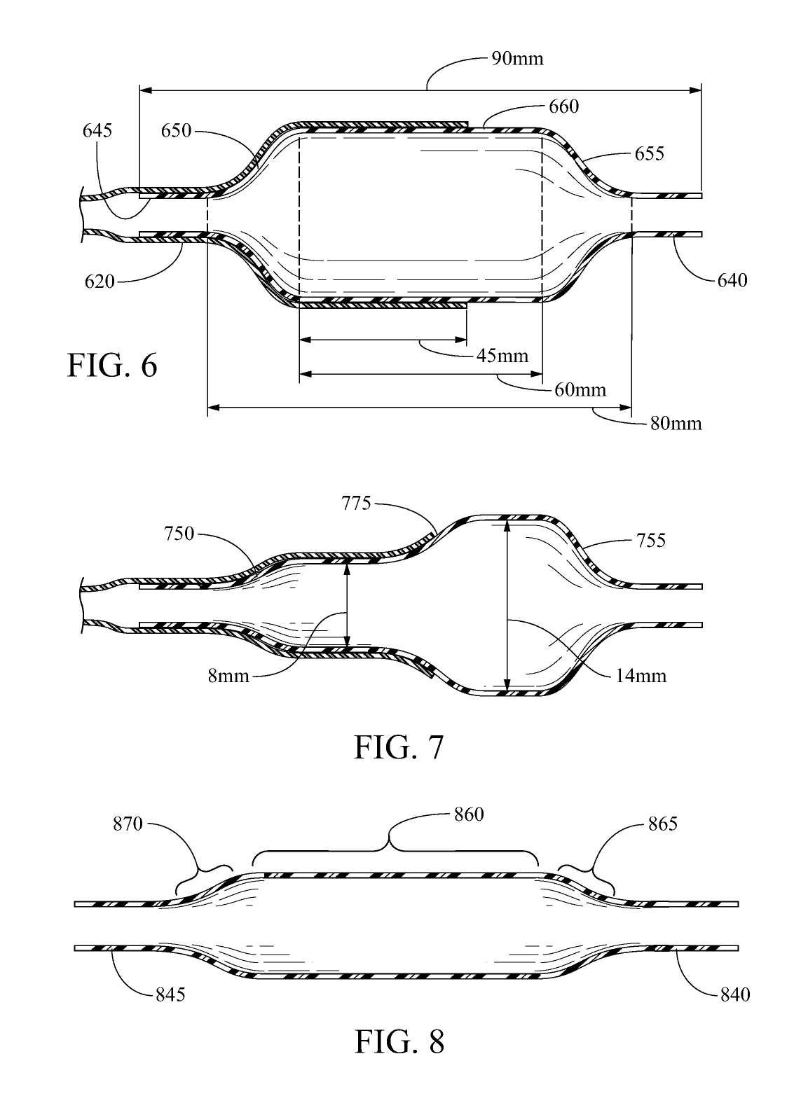

In one embodiment, as can be seen in FIG. 6, the total length of the balloon may be about 90 mm. In this example, the length of the distal neck/leg (640) of the balloon may be about 5 mm and the length of the proximal neck/leg (645) may be about 5 mm. The length of the proximal cone (650) may be about 10 mm and the length of the distal cone (655) may be about 10 mm. The body portion of the balloon (660) or the portion between the proximal and distal cones may be about 60 mm. The length of the sleeve (620) may be appropriately adjusted depending upon how much of the balloon is desired to be covered. In FIG. 6, the length of the sleeve (620) is about 60 mm. However, with respect to this specific example, if only the proximal portion of the balloon (excluding the proximal neck) and a portion of the body portion of the balloon were desired to be covered, the length of the sleeve would be from about 15 mm to about 70 mm.

If a smaller portion of the body portion of the balloon is to be covered in addition to a proximal or distal cone, the length of the sleeve may be from about 15 mm to about 55 mm or from about 15 mm to about 30 mm. If two sleeves are used and a portion of the body portion of the balloon is to remain uncovered by the sleeves, the length of each sleeve can be independently selected from about 10 mm to about 40 mm, if the total length of the balloon is about 90 mm. Again, the foregoing lengths of the various components of the presently disclosed medical devices are for illustrative purposes only and any lengths may be selected depending upon the desired application.

While the presently disclosed balloon may comprise one or more sleeves on one or more portions of the balloon, and one or more portions of the balloon may remain uncovered by the one or more sleeves, specific configurations will now be disclosed. The following configurations are meant as illustrative examples and the present disclosure is not limited to the following configurations.

In one embodiment, the body portion of the balloon comprises a sleeve and the distal and proximal end portions of the balloon remain uncovered by the sleeve. In another embodiment, a distal end portion of the balloon comprises the sleeve and the body portion and proximal end portion of the balloon remain uncovered by the sleeve. In a further embodiment, the proximal end portion of the balloon comprises the sleeve and the body portion and distal end portion of the balloon remain uncovered by the sleeve. In an additional embodiment, the distal end portion of the balloon comprises the sleeve, the proximal end portion of the balloon comprises a second sleeve, and the body portion of the balloon remains uncovered. In another embodiment, the distal end portion of the balloon and at least a section of the body portion of the balloon comprise the sleeve and the proximal end portion of the balloon remains uncovered. In a further embodiment, the proximal end portion of the balloon and at least a section of the body portion of the balloon comprise the sleeve and the distal end portion of the balloon remains uncovered. In any of these configurations, a stent may be disposed on one or more of the sleeves and/or one or more uncovered portions of the balloon.

In one particular embodiment, a system for modifying the behavior of a vascular balloon is disclosed. The system incorporates a catheter comprising a shaft having a distal end and a proximal end. An expandable balloon is mounted at the distal end of the shaft. The shaft has a lumen extending therethrough and in fluid communication with an interior region of the balloon. The balloon comprises a body portion, a distal end portion, and a proximal end portion. A tubular sleeve is disposed around the balloon but at least a portion of the distal end portion, proximal end portion, or body portion of the balloon remains uncovered by the sleeve. The sleeve is adhered to the balloon. The distal end portion of the balloon and the proximal end portion of the balloon are portions that do not include the leg/neck portions of the balloon, which are attached to the catheter. In some aspects, a thickness of a wall of the sleeve is varied along its length.

In another specific embodiment, a system for modifying the behavior of a vascular balloon is disclosed. The system includes a catheter comprising a shaft having a distal end and a proximal end. An expandable balloon is mounted at the distal end of the shaft and the shaft has a lumen extending therethrough and in fluid communication with an interior region of the balloon. The balloon comprises a body portion, a distal end portion, and a proximal end portion. The distal end portion of the balloon and the proximal end portion of the balloon are portions that do not include the leg/neck portions of the balloon, which are attached to the catheter. A tubular sleeve is disposed around the distal end portion of the balloon and a second tubular sleeve is disposed around the proximal end portion of the balloon. In this configuration, the body portion of the balloon remains uncovered. Further, at least one of the tubular sleeve and the second tubular sleeve is adhered to the balloon. In some aspects, the first and second tubular sleeves are both adhered to the balloon. In some aspects, a thickness of a wall of at least one of the tubular sleeve and the second tubular sleeve is varied along its length.

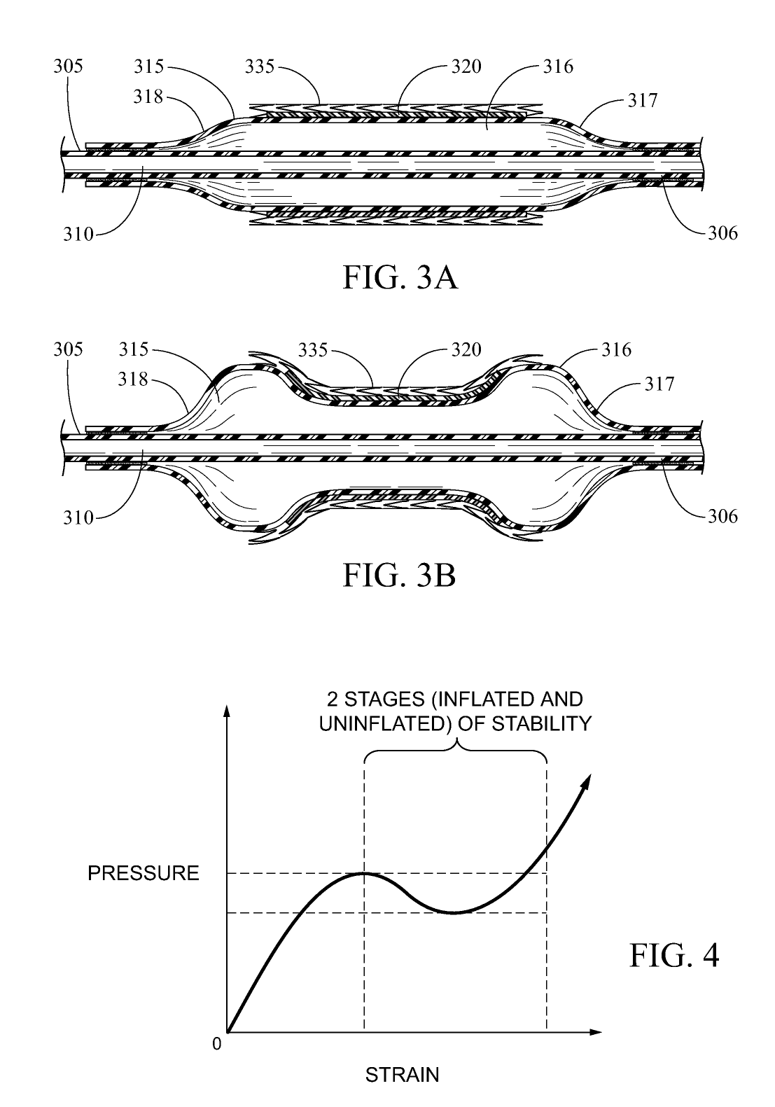

In another specific embodiment, such as that shown in FIGS. 3A and 3B, a system for modifying the behavior of a vascular balloon is disclosed. The system comprises a catheter having a shaft (305) including a distal end (306) and a proximal end (not shown). An expandable balloon (315) is mounted at the distal end of the shaft and the shaft has a lumen (310) extending therethrough and in fluid communication with an interior region of the balloon. The balloon comprises a body portion (316), a distal end portion (317), and a proximal end portion (318). A tubular sleeve (320) is disposed around the body portion of the balloon. The distal portion of the balloon and the proximal portion of the balloon remain uncovered by the sleeve. The distal end portion of the balloon and the proximal end portion of the balloon are portions that do not include the leg/neck portions of the balloon, which are attached to the catheter. In some aspects, a stent (335) may be disposed on the sleeve and/or balloon. In some aspects, a thickness of a wall of the sleeve is varied along a length of the sleeve. In some aspects, the tubular sleeve is adhered to the balloon. This specific embodiment may be useful if a user wishes to prevent a BX stent from slipping during deployment or if a user wishes to create an hourglass-shaped stent (see FIG. 3B) to reduce flow through a vessel, such as in a TIPS revision procedure.

In a further specific embodiment, a system for modifying the behavior of a vascular balloon is disclosed. The system includes a catheter having a shaft comprising a distal end and a proximal end. An expandable balloon is mounted at the distal end of the shaft and the shaft has a lumen extending therethrough and in fluid communication with an interior region of the balloon. The balloon comprises a body portion, a distal end portion, and a proximal end portion. A tubular sleeve is disposed around the proximal portion of the balloon, wherein the distal portion of the balloon remains uncovered by the sleeve. The distal end portion of the balloon and the proximal end portion of the balloon are portions that do not include the leg/neck portions of the balloon, which are attached to the catheter. In some aspects, a thickness of a wall of the sleeve is varied along a length of the sleeve. In some aspects, the sleeve is adhered to the balloon. Further, is some aspects, the body portion of the balloon is also covered by the sleeve. This embodiment may be useful, for example, if a user wishes to create a “sea anchor” to drift with blood flow into a position, or to allow tactile positioning at a point in the vessel where the diameter changes.

In another specific embodiment, a system for modifying the behavior of a vascular balloon is disclosed. The system comprises a catheter including a shaft having a distal end and a proximal end. An expandable balloon is mounted at the distal end of the shaft and the shaft has a lumen extending therethrough and in fluid communication with an interior region of the balloon. The balloon comprises a body portion, a distal end portion, and a proximal end portion. A tubular sleeve is disposed around the distal portion of the balloon, wherein the proximal portion of the balloon remains uncovered by the sleeve. In some aspects, the body portion of the balloon is also covered by the sleeve. The distal end portion of the balloon and the proximal end portion of the balloon are portions that do not include the leg/neck portions of the balloon, which are attached to the catheter. In some aspects, a thickness of a wall of the sleeve is varied along a length of the sleeve. In some aspects, the sleeve is adhered to the balloon. This embodiment may be useful, for example, if a user wishes to create a “sea anchor” to drift with blood flow into a position, or to allow tactile positioning at a point in the vessel where the diameter changes.

In addition to the foregoing benefits of the presently disclosed sleeve, if a balloon should burst during expansion or otherwise during a medical procedure, the content of the balloon may be held in place by the sleeve, thereby creating a safety feature.

All of the compositions, balloons, sleeves, and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a sleeve” is intended to include “at least one sleeve” or “one or more sleeves.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A system for modifying the behavior of a vascular balloon comprising:
a shaft having a distal end and a proximal end;
an expandable balloon mounted at the distal end of the shaft, the shaft having a lumen extending therethrough and in fluid communication with an interior region of the balloon, wherein the balloon comprises a body portion, a distal end portion, and a proximal end portion;
a tubular sleeve disposed around the balloon, wherein a portion of the balloon remains uncovered by the sleeve and wherein the sleeve is adhered to the balloon.

2. The system of claim 1, wherein a portion of the sleeve comprises a thinner wall thickness and a portion of the sleeve comprises a thicker wall thickness, further wherein the portion having the thinner wall thickness is less resistant to opening pressure than the portion having a thicker wall thickness.

3. The system of claim 1, wherein the thickness of the wall of the sleeve is from about 0.005 inches to about 0.015 inches.

4. The system of claim 2, wherein the portion of the sleeve having the thinner wall thickness comprises a thickness from about 0.005 inches to less than about 0.01 inches and the portion of the sleeve having the thicker wall thickness comprises a thickness from greater than about 0.01 inches to about 0.015 inches.

5. The system of claim 1, wherein the sleeve is radially expandable and/or longitudinally expandable.

6. The system of claim 1, wherein the body portion comprises the sleeve and the distal and proximal end portions remain uncovered by the sleeve.

7. The system of claim 1, wherein the distal end portion of the balloon comprises the sleeve and the body portion and proximal end portion remain uncovered by the sleeve.

8. The system of claim 1, wherein the proximal end portion of the balloon comprises the sleeve and the body portion and distal end portion remain uncovered by the sleeve.

9. The system of claim 1, wherein the distal end portion of the balloon comprises the sleeve, the proximal end portion of the balloon comprises a second sleeve, and the body portion of the balloon remains uncovered.

10. The system of claim 1, wherein the distal end portion of the balloon and the body portion of the balloon comprise the sleeve and the proximal end portion of the balloon remains uncovered.

11. The system of claim 1, wherein the proximal end portion of the balloon and the body portion of the balloon comprise the sleeve and the distal end portion of the balloon remains uncovered.

12. The system of claim 1, wherein the sleeve comprises a distal portion, a proximal portion, and a body portion, further wherein at least one portion of the sleeve comprises a different material or different combination of materials than the other portions of the sleeve.

13. The system of claim 1, wherein a stent is disposed on the sleeve and/or the uncovered portion of the balloon.

14. A system for modifying the behavior of a vascular balloon comprising:
a shaft having a distal end and a proximal end;
an expandable balloon mounted at the distal end of the shaft, the shaft having a lumen extending therethrough and in fluid communication with an interior region of the balloon, wherein the balloon comprises a body portion, a distal end portion, and a proximal end portion;
a tubular sleeve disposed around the body portion of the balloon, wherein the distal portion of the balloon and the proximal portion of the balloon remain uncovered by the sleeve.

15. The system of claim 14, wherein the tubular sleeve is adhered to the balloon.

16. The system of claim 14, wherein the tubular sleeve is radially expandable and/or longitudinally expandable.

17. The system of claim 14, wherein the tubular sleeve comprises a distal portion, a proximal portion, and a body portion, further wherein at least one portion of the tubular sleeve comprises a different material or different combination of materials than the other portions.

18. A system for modifying the behavior of a vascular balloon comprising:
a shaft having a distal end and a proximal end;
an expandable balloon mounted at the distal end of the shaft, the shaft having a lumen extending therethrough and in fluid communication with an interior region of the balloon, wherein the balloon comprises a body portion, a distal end portion, and a proximal end portion; and
a tubular sleeve disposed around the proximal portion of the balloon, wherein the distal portion of the balloon remains uncovered by the sleeve; or
a tubular sleeve disposed around the distal portion of the balloon, wherein the proximal portion of the balloon remains uncovered by the sleeve.

19. The system of claim 18, wherein at least a portion of the body portion of the balloon is covered by the sleeve.

20. The system of claim 18, wherein the sleeve is adhered to the proximal portion of the balloon or the distal portion of the balloon.
